(19) United States
(12) Patent Application Publication
(10) Pub. No.: US 2016/0262691 A1
(43) Pub. Date: Sep. 15, 2016

(54) METHOD AND SYSTEM FOR PAIN MONITORING AND MANAGEMENT IN PEDIATRIC PATIENTS

(71) Applicants: LAKSHYA JAIN, LOS ALTOS, CA (US); PRIYANKA BISARYA, SAN DIEGO, CA (US)

(72) Inventors: LAKSHYA JAIN, LOS ALTOS, CA (US); PRIYANKA BISARYA, SAN DIEGO, CA (US)

(21) Appl. No.: 14/616,225

(22) Filed: Feb. 6, 2015

(51) Int. Cl. A61B 5/00; A61B 5/024; A61B 5/053; A61B 5/08; A61B 5/021; A61B 5/0496; A61B 5/0402; A61B 5/0476; A61B 5/0488; A61B 5/11; A61B 5/0408; A61B 5/16; A61B 5/0205; A61B 5/1455

(52) U.S. Cl. CPC A61B 5/4827; A61B 5/0205; A61B 5/02405; A61B 5/053; A61B 5/08; A61B 5/021; A61B 5/14552; A61B 5/0402; A61B 5/0476; A61B 5/0488; A61B 5/1118; A61B 5/6826; A61B 5/0408; A61B 5/04085; A61B 5/165; A61B 5/168; A61B 5/02416; A61B 5/024; A61B 5/1102; A61B 5/0496; A61B 5/0533; A61B 5/7264; A61B 5/0022; A61B 2503/06; A61B 2503/04

(57) ABSTRACT

The present disclosure provides a method and system for monitoring intensity of pain experienced by one or more users. The one or more users are pediatric patients. The method includes assessing the intensity of the pain experienced by each of the one or more users on one or more pain monitoring scales by using one or more bio-markers, fetching the one or more bio-markers associated with each of the one or more users by a plurality of bio-sensors, determining a co-relation between the one or more bio-markers and the intensity of pain experienced by each of the one or more users and generating a pain profile of each of the one or more users.

### TECHNICAL FIELD

The present invention relates to the field of biomedical technology and, in particular, relates to monitoring pain of pediatric patients to tailor treatments accordingly.

### BACKGROUND

Pain is an unpleasant sensory and emotional experience associated with actual or potential tissue damage. In fact, the pain is a stressor and environment challenge that requires the organism to respond. It is a specific emotion caused by a stimulus that reflects homeostatic behavioral drive similar to temperature, itching, hunger, thirst and the like. However, pain sensation may not be necessarily dependent on the tissue damage; it may be generated by the conditional stimuli capable of eliciting strong affective response including sound of a drill, a gentle touch of needle during injections and the like. The pain may be categorized according to various factors including type of damage, time for healing and the like. On the basis of healing, the pain can be categorized as a chronic pain and an acute pain. The chronic pain lasts for a longer time as compared to the acute pain. However, both the chronic pain and the acute pain are extremely important problems leading to loss of working capabilities, financial resources and the like.

Moreover, other categories of the pain include nociceptive pain and neuropathic pain. The nociceptive pain further includes visceral and somatic pain, and neuropathic pain further includes peripheral and central neuropathic pain. The nociceptive pain is the discomfort experienced as a result of an injury. The injury may include but not be limited to a paper cut, a broken bone, appendicitis and the like.

The neuropathic pain is associated with an injury to a nerve or central nervous system. Such injuries can give rise to paresthesias. For example, the paresthesias may include but not be limited to numbness, tingling, electrical sensations and the like. Further, the neuropathic pain can also generate unusual symptoms. For example, the unusual symptoms may include anesthesia dolorosa in which area producing the pain is numb to touch.

Experience of the pain varies from person to person due to inter-individual variability. Moreover, intensity of the pain varies from cause to cause in the individual. Thus, pain management is an extremely important issue. Various factors, directly or indirectly, contributes in controlling the pain. For example, biological factors (for e.g., gender, genetics and the like), psychological factors (for e.g., mood, attention, distraction and the like), social factors (for e.g., marital status, social support and the like) and the like can significantly modulate the intensity as well as unpleasantness caused by the pain.

Despite many advances in pain management field, a significant number of pediatric population experience pain unnecessarily in medical settings. Recent data have shown that approximately 20-25% of pediatric patients in hospitals experience unnecessary pain. Further, addiction and misuse of prescribed drugs is a major problem with the pediatric population. Appropriate pain management and treatment of children in pain includes reduction of stress and anxiety, use of appropriate topical IV, transdermal, intranasal and oral pain medications and prevention of pain with procedures. An administration of a 12% to 25% solution of sucrose can help reduce neonatal distress during painful procedures in the pediatric population. The sucrose decreases the response to noxious stimuli including heel sticks and injections and reduces subsequent crying episodes during routine care of neonates. In addition, use of pacifiers, either with or without sucrose reduces neonatal distress.

Presently, it is known that functional interactions exist between systems controlling cardiovascular functions and systems modulating perception of the pain. In addition, there are relationships between pain stimuli and autonomic reactions. Some changes happen in body due to reaction of autonomic nervous system (hereinafter ‘ANS’) to the pain. For example, blood pressure (hereinafter ‘BP’), heart rate (hereinafter ‘HR’), heart rate variability (hereinafter ‘HRV’) and the like. These interactions are important for diagnosing and regulating the pain. For example, the HRV can be used as an important indicator of the ANSreactivity to nociceptive stimulation. Some changes happen due to response of somatic nervous system to the pain which may include fidgeting or moving some limb of body or any other type of physical movement under pain. However, presently, there is no method and system that studies these relationships and the interactions to guide appropriate treatment, care, lifestyle and the like for healthy response in the individuals with respect to the pain. Further, there is no method and system to monitor the intensity of the pain experienced by the pediatric population.

In light of the above stated discussion, there is a need for a method and system that overcomes the above stated disadvantages. In addition, the method and system should monitor the pain of the pediatric patients and enable tailoring of treatments accordingly. Further, the method and system should be able to monitor both the somatic pain and the neuropathic pain whose effect may manifest differently in the body.

### SUMMARY

In an aspect of the present disclosure, a method and system for monitoring intensity of pain experienced by one or more users is provided. The one or more users are pediatric patients. The method includes assessing the intensity of the pain experienced by each of the one or more users on one or more pain monitoring scales by using one or more bio-markers, fetching the one or more bio-markers associated with each of the one or more users by a plurality of bio-sensors, determining a co-relation between the one or more bio-markers and the intensity of the pain experienced by each of the one or more users and generating a pain profile of each of the one or more users. The generated pain profile shows the intensity of pain experienced by each of the one or more users at various points in body aiding in better medical treatment of each of the one or more users.

In an embodiment of the present disclosure, the one or more pain monitoring scales include scales for measuring pain of one or more neonates, one or more infants or one or more toddlers. For example, the scales can be a neonatal pain agitation and sedation scale (N-PASS), a pain assessment tool (PAT), a bernese pain scale for one or more neonates (BPSN), wong-baker scale, a face, legs, activity, crying, and consolability (FLACC) scale, an expert physician's quantified pain scale and the like. An expert physician can rate the pain of child based on each of the one or more pain monitoring scales or quantify it based on his own assessment. Further, the method includes recording the one or more bio-markers for each state in ranking of each scale of the one or more pain monitoring scales. Furthermore, the method includes mimicking an opinion of the expert physician for each scale. Moreover, a non-expert can find or judge the pain of the one or more neonates, the one or more infants or the one or more toddlers per each of the one or more pain monitoring scales based on readings of the one or more bio-markers. It should be noted that if even one scale of the one or more pain monitoring scales indicates pain, then the one or more neonates, one or more infants or one or more toddlers are declared to have pain. In addition, information judged by the non-expert can be communicated to a more experienced nurse, caretaker or a physician who is better versed with interpreting these scales and with the treatment that should be given to the one or more users based on these scales.

In another embodiment of the present disclosure, the one or more pain monitoring scales include a visual analog scale (VAS), a verbal numerical rating scale (VNRS), a brief pain inventory (BPI), verbal descriptor scale (VDS), the expert physician's quantified pain scale and the like. The one or more pain monitoring scales rank the intensity of the pain experienced by pediatric population who are mature enough to interpret perception of the pain on each of the one or more pain monitoring scales. Each of the pain monitoring scales rate the intensity of the pain, say from values 1 to 10. In an embodiment of the present disclosure, the value 10 can represent the highest pain level experienced by the pediatric population. The expert physician can rate the pain of the child according to each of these multiple pain scales. Further, the method includes recording the one or more bio-markers for each state in ranking of the each scale. Furthermore, the method includes mimicking an opinion of the expert physician for each scale. Moreover, a non-expert can find or judge the pain of the one or more children per each of the one or more pain monitoring scales based on readings of the one or more bio-markers. In addition, information judged by the non-expert can be communicated to a more experienced nurse, caretaker or a physician who is better versed with interpreting these scales and with the treatment that should be given to the one or more users based on these scales.

In an embodiment of the present disclosure, the one or more bio-markers associated with each of the one or more users includes heart rate (HR), heart rate variability (HRV), skin conductance, respiration information, blood pressure, photoplethysmography (PPG), oxygen saturation, single or multiple lead electrocardiography (ECG), electroencephalography (EEG), muscle activity (EMG), pulse wave transit time, atrial kick, BCG (Balistocardiogram), EOG (Electrooculography), Dispersion based ECG, Impedence cardiography, GSR, VO₂ max, PaCO₂, facial features, stress, emotion detectors, cardiac output, oxygen saturation, blood glucose, blood gas, temperature, sweat, hydration, gaze, movements, and restlessness.

In an embodiment of the present disclosure, the plurality of bio-sensors includes a finger based pulse oximeter, an accelerometer, a respiration monitor and a 1-lead disposable electrocardiography (ECG) patch or a multiple lead ECG.

In an embodiment of the present disclosure, the pain profile for each of the one or more users is generated using any combination of the plurality of bio-sensors.

In an embodiment of the present disclosure, the generated pain profile for each of the one or more users utilizes a pre-defined color coding or pain quantification scale or any other visual approach that can indicate the level of pain, based on the intensity and location of the pain in the body of each of the one or more users. A change in intensity of colors or pain quantification or indication given by any other visual approach adopted is directly proportional to the intensity of the pain experienced by the one or more users. Even though, the child may not be able to explain the intensity of the pain, the physician is able to quantify the pain by touching one or more regions of the pain, singly or in combination, and noting/observing reaction of the child. Further, the readings of the one or more bio-markers of the pediatric population are associated with the physician's assessment to train the machine learning model. The machine learning model trained by the physician may be utilized by the non-expert to quantify the pain using the machine learning model in the same way as the expert physician would, and dispense the treatment accordingly.

In an embodiment of the present disclosure, the method distributes the one or more users into different sets based on their phenotypical characteristics, genotypical characteristics and mental attributes.

In an embodiment of the present disclosure, the intensity of pain experienced by the one or more users is characterized by at least one of biological factors (gender, genetics and the like), psychological factors (mood, attention and the like), experimental factors, duration of measurement of the intensity of the pain and location of each sensor of the plurality of bio-sensors on the body of each of the one or more users.

In an embodiment of the present disclosure, the method tracks the location of each of the plurality of bio-sensors on the body of each of the one or more users for the monitoring of the intensity of pain experienced by each of the one or more users.

In another aspect of the present disclosure, a system for monitoring intensity of pain experienced by one or more users is provided. The one or more users are pediatric patients. The system includes a plurality of bio-sensors to fetch one or more bio-markers associated with each of the one or more users and a pain monitoring application. The pain monitoring application further includes an input/output module to fetch the one or more bio-markers associated with each of the one or more users, a display module to display the one or more bio-markers associated with the one or more users, a diagnostic module to assess the one or more bio-markers of each of the one or more users to determine the intensity and location of the pain, a presentation module to generate a pain profile for each of the one or more users and a database to store the fetched plurality of the one or more bio-markers associated with each of the one or more users and the generated pain profile for each of the one or more users. The generated pain profile shows the intensity of pain experienced by the one or more users at various points in the body aiding in better treatment of the one or more users.

In an embodiment of the present disclosure, the one or more pain monitoring scales include scales for measuring pain of one or more neonates, one or more infants or one or more toddlers. For example, scales can be a neonatal pain agitation and sedation scale (N-PASS), a pain assessment tool (PAT), a bernese pain scale for one or more neonates (BPSN), wong-baker scale, a face, legs, activity, crying, and consolability (FLACC) scale, an expert physician's quantified pain scale and the like. Each of the one or more pain monitoring scales rank the intensity of pain experienced by the one or more neonates, the one or more infants and the one or more toddlers. An expert physician can rate the pain of child based on each of theone or more pain monitoring scales. Further, the method includes recording the one or more bio-markers for each state in ranking of each scale. Furthermore, the method includes mimicking an opinion of the expert physician for each scale. Moreover, a non-expert can find or judge the pain of the one or more neonates, the one or more infants or the one or more toddlers per each of the one or more pain monitoring scales based on readings of the one or more bio-markers. It should be noted that if even one scale of the one or more pain monitoring scales indicates pain, then the one or more neonates, one or more infants or one or more toddlers are declared to have pain. In addition, information judged by the non-expert can be communicated to a more experienced nurse, caretaker or a physician who is better versed with interpreting these scales and with the treatment that should be given to the one or more users based on these scales.

In another embodiment of the present disclosure, the one or more pain monitoring scales include a visual analog scale (VAS), a verbal numerical rating scale (VNRS), a brief pain inventory (BPI), verbal descriptor scale (VDS), the expert physician's quantified pain scale and the like. Each of the one or more pain monitoring scales rank the intensity of the pain experienced by pediatric population who are mature enough to interpret perception of the pain on these pain monitoring scale. Each of these pain monitoring scales rate the intensity of the pain, say from values 1 to 10. In an embodiment of the present disclosure, the value 10 can represent the highest pain level experienced by the pediatric population. The expert physician can rate the pain of the child according to each of the one or more pain monitoring scales. Further, the method includes recording the one or more bio-markers for each state in ranking of the each scale. Furthermore, the method includes mimicking an opinion of the expert physician for each scale. Moreover, a non-expert can find or judge the pain of the one or more neonates, the one or more infants or the one or more toddlers per each of the one or more pain monitoring scales based on readings of the one or more bio-markers. In addition, information judged by the non-expert can be communicated to a more experienced nurse, caretaker or a physician who is better versed with interpreting these scales and with the treatment that should be given to the one or more users based on these scales.

In an embodiment of the present disclosure, the diagnostic module further determines co-relation between the one or more bio-markers associated with each of the one or more users and the intensity of pain experienced by each of the one or more users.

In an embodiment of the present disclosure, the diagnostic module further tracks location of each of the plurality of bio-sensors on the body of each of the one or more users for the monitoring of the intensity of pain experienced by each of the one or more users.

In an embodiment of the present disclosure, the fetched one or more bio-markers associated with each of the one or more users include heart rate (HR), heart rate variability (HRV), skin conductance, respiration information, blood pressure, photoplethysmography (PPG), oxygen saturation, single or multiple lead electrocardiography (ECG), electroencephalography (EEG), muscle activity (EMG), pulse wave transit time, atrial kick, BCG (Balistocardiogram), EOG (Electrooculography), Dispersion based ECG, Impedence cardiography, GSR, VO₂ max, PaCO₂, facial features, stress, emotion detectors, cardiac output, oxygen saturation, blood glucose, blood gas, temperature, sweat, hydration, gaze, movements, and restlessness.

In an embodiment of the present disclosure, the generated pain profile for each of the one or more users utilizes a pre-defined color coding or pain quantification scale or any other visual approach that can indicate the level of pain based on the intensity and location of the pain in the body of each of the one or more users. A change in intensity of colors or pain quantification scale or indication given by any other visual approach adopted is directly proportional to the intensity of the pain experienced by the one or more users. Even though, the child may not be able to explain the intensity of the pain, the physician is able to quantify the pain by touching one or more regions of the pain, singly or in combination, and noting/observing reaction of the child. Further, the readings of the one or more bio-markers of the pediatric population are associated with the physician's assessment to train the machine learning model. The machine learning model trained by the physician may be utilized by the non-expert to quantify the pain using the machine learning model in the same way as the expert physician would, and dispense the treatment accordingly.

In an embodiment of the present disclosure, the intensity of the pain experienced by the one or more users is characterized by biological factors (gender, genetics and the like), psychological factors (mood, attention and the like), experimental factors, duration of measurement of the intensity of the pain and the location of each sensor of the plurality of bio-sensors on the body of each of the one or more users.

In yet another aspect of the present disclosure, a computer system is provided. The computer system includes one or more processors and a non-transitory memory containing instructions that, when executed by the one or more processors, causes the one or more processors to perform a set of steps. The set of steps includes assessing intensity of pain experienced by each of one or more users on one or more pain monitoring scales by using one or more bio-markers, fetching the one or more bio-markers associated with each of the one or more users by a plurality of bio-sensors, determining a co-relation between the one or more bio-markers and the intensity of pain experienced by each of the one or more users and generating a pain profile of each of the one or more users. The generated pain profile shows the intensity of pain experienced by each of the one or more users at various points in body aiding in better medical treatment of each of the one or more users.

In an embodiment of the present disclosure, the non-transitory memory containing instructions that, when executed by the one or more processors, cause the one or more processors to perform a further step of determining co-relation between theone or more bio-markers associated with each of the one or more users and the intensity of the pain experienced by each of the one or more users.

### BRIEF DESCRIPTION OF THE FIGURES

Having thus described the invention in general terms, reference will now be made to the accompanying drawings, which are not necessarily drawn to scale, and wherein:

FIG. 1 illustrates a system showing an interaction among various components for monitoring intensity of pain experienced by one or more users, in accordance with various embodiments of the present disclosure;

FIG. 2 illustrates a system showing a block diagram of a communication device , in accordance with various embodiments of the present disclosure;

FIG. 3 illustrates a flow chart for monitoring the intensity of the pain experienced by the one or more users, in accordance with the various embodiments of the present disclosure; and

FIG. 4 illustrates a block diagram of a communication device, in accordance with various embodiments of the present disclosure.

### DETAILED DESCRIPTION

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

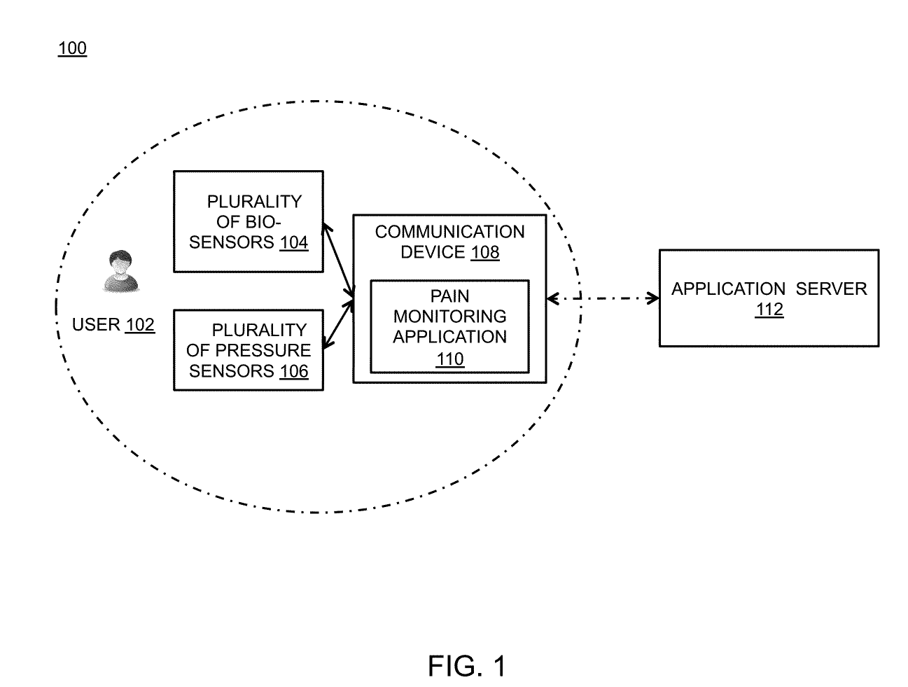

FIG. 1 illustrates a system 100 showing interaction among various components for monitoring intensity of pain experienced by one or more users, in accordance with various embodiments of the present disclosure. The one or more users are pediatric patients. The system 100 includes a plurality of bio-sensors 104, a plurality of pressure sensors 106 and a communication device 108 associated with a user 102. Examples of the communication device 108 include but may not be limited to mobile phone, laptop, desktop computer and the like. The communication device 108 executes a pain monitoring application 110. The pain monitoring application 110 monitors the pain of the user 102 and allows tailoring of treatments accordingly. The pain monitoring application 110 communicates with an application server 112 via a network. The application server 112 runs the pain monitoring application 110. The user 102 may be a healthy individual or a patient suffering from the pain. The plurality of bio-sensors 104 captures one or more bio-markers associated with the user 102. The one or more bio-markers includes heart rate (hereinafter ‘HR’), blood pressure (hereinafter ‘BP’), respiratory information, skin conductance and the like. The plurality of pressure sensors 106 determines sensitivity of the pain by pressurizing areas of patient's body (body of the user 102) at which the pain is to be diagnosed.

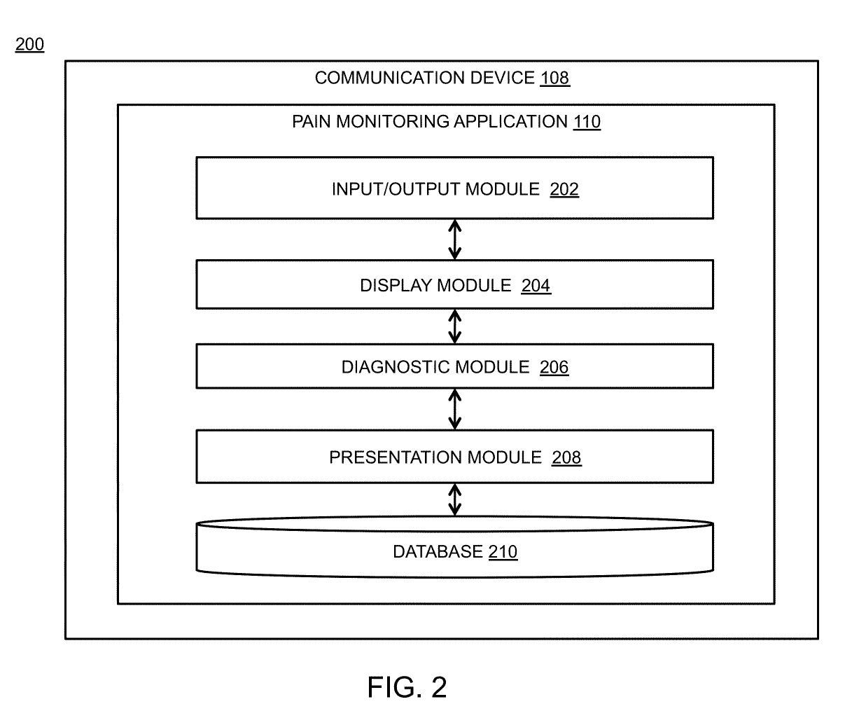

FIG. 2 illustrates a system 200 showing a block diagram of the communication device 108, in accordance with various embodiments of the present disclosure. The communication device 108 executes the pain monitoring application 110. The pain monitoring application 110 analyzes the intensity and area of the pain and model the pain to enable tailoring of the treatments accordingly. The pain monitoring application 110 includes an input/output module 202, a display module 204, a diagnostic module 206, a presentation module 208 and a database 210. The input/output module 202 receives the one or more bio-markers from the plurality of bio-sensors 104 associated with the user 102. The display module 204 displays the received plurality of the one or more bio-markers associated with the user 102.

The diagnostic module 206 assesses the intensity of the pain of the user 102 on one or more pain monitoring scales that include one or more scales for measuring the pain of the user 102. The user 102 can be one or more neonates, one or more infants or one or more toddlers. The scales can be a neonatal pain agitation and sedation scale (N-PASS), a pain assessment tool (PAT), a bernese pain scale for one or more neonates (BPSN), wong-baker scale, a face, legs, activity, crying, and consolability (FLACC) scale, an expert physician's quantified pain scale and the like. Each of the one or more pain monitoring scales rank the intensity of pain experienced by the one or more neonates, the one or more infants and the one or more toddlers.

An expert physician can rate the pain of the user 102 based on each of these multiple pain scales. Further, the method includes recording the one or more bio-markers for each state in ranking of the each scale. Furthermore, the method includes mimicking an opinion of the expert physician for each scale. Moreover, a non-expert can find or judge the pain of the one or more neonates, the one or more infants or the one or more toddlers per each of the one or more pain monitoring scales based on readings of the one or more bio-markers. It should be noted that if even one scale of the one or more pain monitoring scales indicates pain, then the user 102 is declared to have pain. In addition, information judged by the non-expert can be communicated to a more experienced nurse, care taker or a physician who is better versed with interpreting these scales and with the treatment that should be given to the one or more users based on these scales.

In an embodiment of the present disclosure, the user 102 is reported to be experiencing the pain by the physician if the readings of any of the one or more bio-markers and/or the one or more pain monitoring scales increase beyond a threshold value.

In another embodiment of the present disclosure, the one or more pain monitoring scales include a visual analog scale (VAS), a verbal numerical rating scale (VNRS), a brief pain inventory (BPI), a verbal descriptor scale (VDS), the expert physician's quantified pain scale and the like. Each of the one or more pain monitoring scales rank the intensity of the pain experienced by pediatric population who are mature enough to interpret perception of the pain on the one or more pain monitoring scales. Each of these pain monitoring scales rate the intensity of the pain, say from values 1 to 10. In an embodiment of the present disclosure, the value 10 can represent the highest pain level experienced by the pediatric population. The expert physician can rate the pain of the pediatric population according to each of these multiple pain scales. Further, the method includes recording the one or more bio-markers for each state in ranking of the each scale. Furthermore, the method includes mimicking an opinion of the expert physician for each scale. Moreover, a non-expert can find or judge the pain of the one or more children per each of the one or more pain monitoring scales based on readings of the one or more bio-markers. It should be noted that if even one scale of the one or more pain monitoring scales indicates pain, then the user 102 is declared to have pain. In addition, information judged by the non-expert can be communicated to a more experienced nurse, care taker or a physician who is better versed with interpreting these scales and with the treatment that should be given to the one or more users based on these scales.

In an embodiment of the present disclosure, the pediatric population is reported to be experiencing the pain by the physician if the readings of any of the one or more bio-markers and/or the one or more pain monitoring scales increase beyond a threshold value.

In addition, the diagnostic module 206 determines a co-relation between the one or more bio-markers and the intensity of the pain of the user 102.

Furthermore, the diagnostic module 206 tracks location of each of the plurality of bio-sensors 104 and each of the plurality of pressure sensors 106 on the body of the user 102. In an embodiment of the present disclosure, the diagnostic module 206 tracks each of the location of each of the plurality of bio-sensors 104 and each of the plurality of pressure sensors 106 as the intensity of the pain experienced by the user 102 is characterized by the location of measuring site. In another embodiment of the present disclosure, the diagnostic module 206 tracks multiple measuring sites for the neonates as the neonates lack ability to communicate the location of the body experiencing the pain. Further, the diagnostic module 206 combines signal values representing pain from the multiple sites of the body to create a composite signal. Any disruption or deviation from baseline may be measured by comparing the composite signal from points/sites of the body that generates a clean signal.

The presentation module 208 generates a pain profile of the user 102. The generated pain profile shows the intensity of the pain at various points in body of the user 102 aiding in better medical treatment of the user 102. Moreover, the pain profile is generated by utilizing color codes with respect to the location and the intensity of the pain experienced by the user 102. A change in intensity of the colors is directly proportional to the pain experienced by the user 102. Moreover, the display module 204 displays the generated pain profile of each of the user 102.

The database 210 stores the fetched one or more bio-markers associated with the user 102 and the generated pain profile of the user 102. In addition, the diagnostic module 206 compares experimental results stored in the database 210 with inputs received by the input/output module 202.

In an embodiment of the present disclosure, a user who is not an expert physician or pediatrician may look at the readings of the one or more bio-markers obtained from each of the one or more pain monitoring scales and co-relate the reading with the intensity of the pain of the user 102. In an embodiment of the present disclosure, if a bio-marker of the one or more bio-markers and/or the one or more pain monitoring scales and/or the one or more pain monitoring scales indicates pain, the user 102 is reported to be experiencing the pain.

In an embodiment of the present disclosure, the one or more bio-markers associated with the user 102 includes the HR, heart rate variability (hereinafter ‘HRV’), skin conductance, respiration information, blood pressure, photoplethysmography (hereinafter ‘PPG’), oxygen saturation, electrocardiogram (hereinafter ‘ECG’) analysis, electroencephalogram (hereinafter ‘EEG’) analysis, muscle activity (hereinafter ‘EMG’), restlessness and the like.

In another embodiment of the present disclosure, the one or more bio-markers associated with the user 102 including the HR, the BP, the respiratory information, the skin conductance and the like may be utilized to track the pain and the location of the pain in the body of the user 102. Moreover, variability in the one or more bio-markers from the one or more bio-markers may serve as an indication of the intensity of the pain felt. For example, using an electrocardiography (ECG) with extremely high sampling rate enables modeling and analyzing of minute variations in the ECG morphology.

In yet another embodiment of the present disclosure, using respiration rate as the bio-marker, respiratory distress, frequency and depth can be monitored that indicates user 102 reactivity to the pain. Moreover, an increase in the respiration rate, an increase in shallow breathing and a loss of respiratory rhythm may indicate greater pain. Similarly, reduction in the HRV and elevation in the HR may indicate severity of the pain. The pain may be modeled and mapped by utilizing changes in low frequency (hereinafter ‘LF’) and/or high frequency (hereinafter ‘HF’) spectrum of the heart rate variability (HRV). For example, greater LF (reduced HF) indicates response to pain stimulus. Further, the skin conductance can be used to model and map the pain. For example, greater skin conductance measured by the galvanic skin response (GSR) serves as the bio-marker indicating the greater pain. Moreover, a noticeable and progressively increasing change in certain dimensions of the EEG reflects increasing pain.

In yet another embodiment of the present disclosure, the stated correlation between the one or more bio-markers associated with the user 102 and the intensity of the pain of the user 102 helps a doctor to determine the patient's (user 102) history and response to the pain and treat the patient (user 102) accordingly. For example, a dental hygienist X is deep cleaning a user Y's teeth. If the dental hygienist X finds that on reaching a certain location, the one or more bio-markers from the plurality of the one or more bio-markers suddenly increase significantly then the dental hygienist X can immediately alter her approach and provide more local anesthetic.

In yet another embodiment of the present disclosure, it is shown that a strong relationship exists between a child's (user 102) dental anxiety and successful dental treatment, and also between anxiety and pain. Painful conditions cause fear whereas fear and anxiety increases the amount of perceived pain. The pain due to dental treatment may induce hemodynamic changes in a patient. The hemodynamic changes include change in the blood flow, motion, equilibrium and the like. The anxiety is a cognitive, an emotional and a physical reaction to an anticipation of a threat. The pain and the anxiety triggered by the dental treatment can induce the secretion of endogenous catecholamines. High catecholamines value in blood induces more stress in the body of the user 102. Moreover, when this situation is combined with local anesthetics and vasoconstrictors use, it increases its undesirable effects on cardiovascular system. For example, a physician reports significant increases (5-12 mmHg) in systolic blood pressure in a patient Y subjected to root scaling and planning using anesthesia with a vasoconstrictor.

In yet another embodiment of the present disclosure, the attenuation of stress with anxiolytics or sedation reduces the cardiovascular response associated with the anxiety of the user 102.

In yet another embodiment of the present disclosure, a fact is recognized that most of the sensors from the plurality of bio-sensors 104 and the plurality of pressure sensors 106 are not easily applicable to be used on infants and neonates. For example, the EEG, a beat-by-beat blood pressure using tonometry is used with difficulty on the infants and the neonates. Thus, the present disclosure recognizes that the monitoring method used in creating the pain profile for the infants and the neonates must work with a small set of sensors from the plurality of bio-sensors 104 and the plurality of pressure sensors 106 that are well regarded to be easy to use in the infants and the neonates.

In yet another embodiment of the present disclosure, the plurality of bio-sensors 104 includes a finger based pulse oximeter, an accelerometer, a respiration monitor and a 1-lead disposable ECG patch.

In yet another embodiment of the present disclosure, the pain profile for the user 102 is generated by using any combination of the sensors from the plurality of bio-sensors 104 and the plurality of pressure sensors 106.

In yet another embodiment of the present disclosure, the intensity of the pain is characterized by tremendous inter-individual variability and is different for different persons. It can be controlled by biological factors (gender, genetics and the like), psychological factors (mood, attention and the like) and social factors (marital status), experimental factors and the like. For example, a patient (user 102) who is experiencing a chronic pain may not report same pain level as those who are new to the pain experience.

In yet another embodiment of the present disclosure, the intensity of the pain experienced by the user 102 is further characterized based on time taken to measure the intensity of pain. The time taken to measure the intensity of the pain helps in the monitoring of the pain experienced by the pediatric patient (user 102) due to a greater sensitivity of the pediatric patient (user 102) towards an excitatory stimulus. The excitatory stimulus is a stimulus for passing information from one neuron to other neuron.

In yet another embodiment of the present disclosure, the monitoring of the intensity of the pain experienced by the user 102 is based on circadian factors of the body of the user 102. The circadian factors involve regulation of timing of biological processes performed by the body of the user 102 relative to 24 hour day/night cycle of nature.

In yet another embodiment of the present disclosure, when a high pain is detected by the one or more bio-markers, a flag of high pain is raised. Further, when the user 102 is detected with no pain, then the one or more pain monitoring scales must declare no pain. Furthermore, an identical argument is applicable for all intermediate levels of the pain.

In yet another embodiment of the present disclosure, the treatment of the user 102 is based on his phenotypical characteristics, genotypical characteristics and mental attributes.

In yet another embodiment of the present disclosure, the need for the user 102 to immediately rush to a medical clinic can be avoided to an extent. For example, if the user 102 is declared to be in pain due to one method (for example, using just the PPG bio-sensor) then the system 100 encourages more sensors from the plurality of bio-sensors 104 to be placed on the body so that measurements can be ascertained. This process can be escalated to any combination of the sensors from the plurality of bio-sensors 104 and the plurality of pressure sensors 106.

In yet another embodiment of the present disclosure, an addition of other pain monitoring scale from the one or more pain monitoring scales depends on the individual judgment of the doctor or a medical professional based on the user 102 past history of the treatment.

In yet another embodiment of the present disclosure, many sensors from the plurality of bio-sensors 104 and the plurality of pressure sensors 106 are expensive and not typically used in a normal setting. These sensors include a beat-to-beat measurement device and invasive monitoring methods. The present disclosure allows such measurements to be added to the pain monitoring process to determine the pain even more accurately.

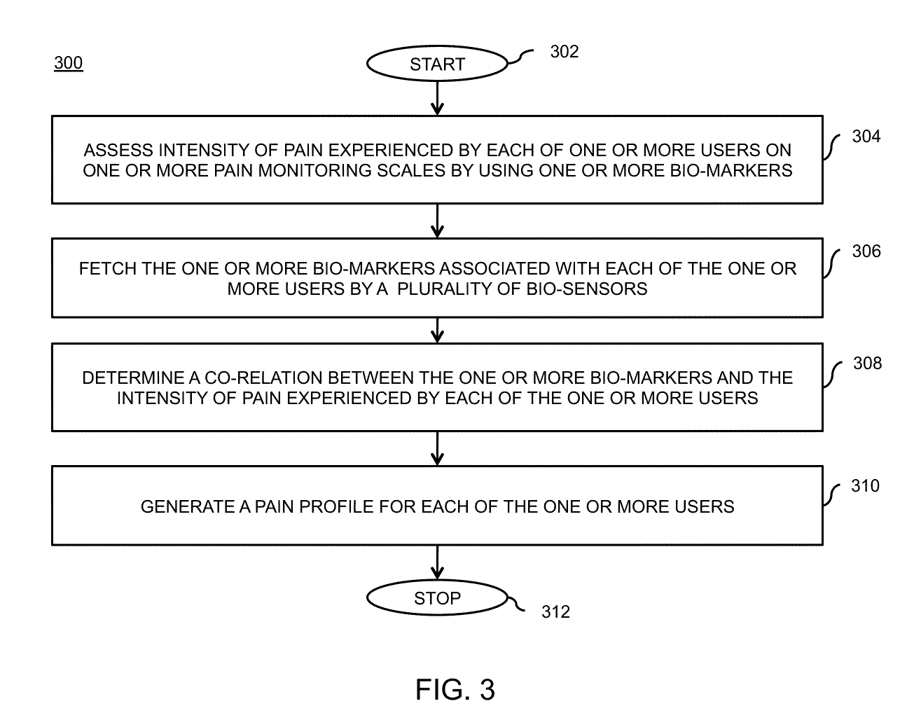

FIG. 3 illustrates a flow chart 300 for monitoring the intensity of the pain experienced by the user 102, in accordance with the various embodiments of the present disclosure. The flow chart initiates at step 302. Following step 302, at step 304, the diagnostic module 206 assesses the intensity of the pain of each of user 102 on one or more pain monitoring scales using the one or more bio-markers. At step 306, the plurality of bio-sensors 104 fetch the one or more bio-markers associated with the user 102. At step 308, the diagnostic module 206 determines a co-relation between the one or more bio-markers and the intensity of the pain of the user 102. At step 310, the presentation module 208 generates a pain profile of the user 102. The flow chart terminates at step 312.

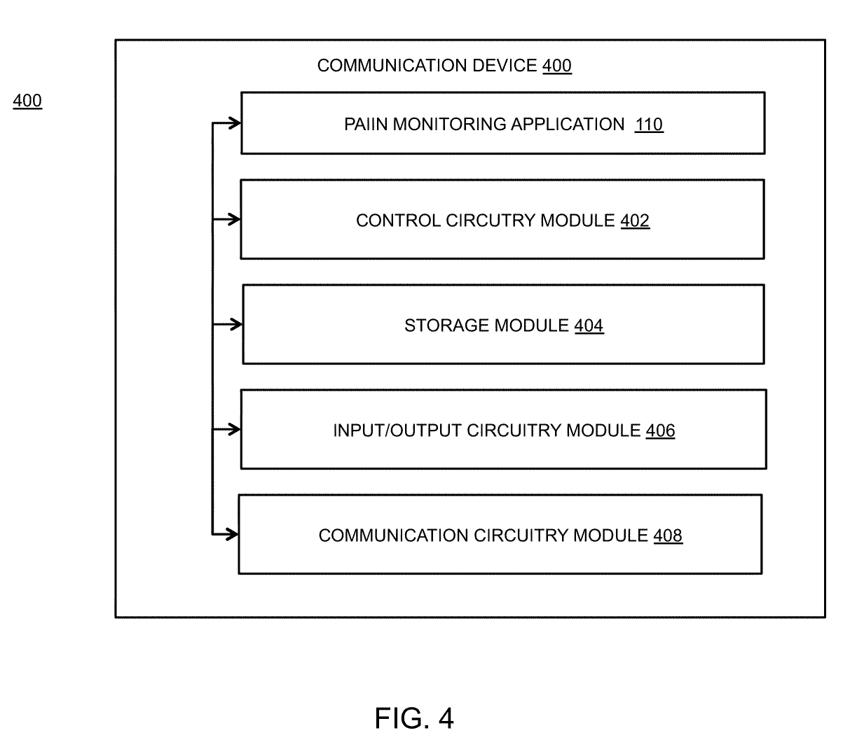

FIG. 4 illustrates a block diagram of a communication device 400, in accordance with various embodiments of the present disclosure. As stated above, in an embodiment, the communication device 400 enables the hosting of the pain monitoring application 110. The communication device 400 includes a control circuitry module 402, a storage module 404, an input/output circuitry module 406 and a communication circuitry module 408. The communication device 400 includes any suitable type of portable electronic device. Examples of the communication device 400 include but may not be limited to a personal e-mail device (e.g., a Blackberry™ made available by Research in Motion of Waterloo, Ontario), a personal data assistant (“PDA”), a cellular telephone, a Smartphone, a handheld gaming device, a digital camera, a laptop computer, and a tablet computer. In another embodiment of the present innovation, the communication device 400 can be a desktop computer.

From the perspective of this innovation, the control circuitry module 402 includes any processing circuitry or processor operative to control the operations and performance of the communication device 400. For example, the control circuitry module 402 may be used to run operating system applications, firmware applications, media playback applications, media editing applications, or any other application. In an embodiment, the control circuitry module 402 drives a display and process inputs received from a user interface.

From the perspective of this innovation, the storage module 404 includes one or more storage mediums including a hard-drive, solid state drive, flash memory, permanent memory such as ROM, any other suitable type of storage component, or any combination thereof. The storage module 404 may store, for example, media data (e.g., music and video files), application data (e.g., for implementing functions on the communication device 400).

From the perspective of this innovation, the I/O circuitry module 406 may be operative to convert (and encode/decode, if necessary) analog signals and other signals into digital data. In an embodiment, the I/O circuitry module 406 may also convert digital data into any other type of signal, and vice-versa. For example, the I/O circuitry module 406 may receive and convert physical contact inputs (e.g., from a multi-touch screen), physical movements (e.g., from a mouse or sensor), analog audio signals (e.g., from a microphone), or any other input. The digital data may be provided to and received from the control circuitry module 402, the storage module 404, or any other component of the communication device 400.

It may be noted that the I/O circuitry module 406 is illustrated in FIG. 4 as a single component of the communication device 400; however those skilled in the art would appreciate that several instances of the I/O circuitry module 406 may be included in the communication device 400.

The communication device 400 may include any suitable interface or component for allowing a user to provide inputs to the I/O circuitry module 406. The communication device 400 may include any suitable input mechanism. Examples of the input mechanism include but may not be limited to a button, keypad, dial, a click wheel, and a touch screen. In an embodiment, the communication device 400 may include a capacitive sensing mechanism, or a multi-touch capacitive sensing mechanism.

In an embodiment, the communication device 400 may include specialized output circuitry associated with output devices such as, for example, one or more audio outputs. The audio output may include one or more speakers built into the communication device 400, or an audio component that may be remotely coupled to the communication device 400.

The one or more speakers can be mono speakers, stereo speakers, or a combination of both. The audio component can be a headset, headphones or ear buds that may be coupled to communications device with a wire or wirelessly.

In an embodiment, the I/O circuitry module 406 may include display circuitry for providing a display visible to the user. For example, the display circuitry may include a screen (e.g., an LCD screen) that is incorporated in the communication device 400.

The display circuitry may include a movable display or a projecting system for providing a display of content on a surface remote from the communication device 400 (e.g., a video projector). In an embodiment, the display circuitry may include a coder/decoder to convert digital media data into analog signals. For example, the display circuitry may include video Codecs, audio Codecs, or any other suitable type of Codec.

The display circuitry may include display driver circuitry, circuitry for driving display drivers or both. The display circuitry may be operative to display content. The display content can include media playback information, application screens for applications implemented on the electronic device, information regarding ongoing communications operations, information regarding incoming communications requests, or device operation screens under the direction of the control circuitry module 402. Alternatively, the display circuitry may be operative to provide instructions to a remote display.

In addition, the communication device 400 includes the communications circuitry module 408. The communications circuitry module 408 may include any suitable communications circuitry operative to connect to a communications network and to transmit communications (e.g., voice or data) from the communication device 400 to other devices within the communications network. The communications circuitry 408 may be operative to interface with the communications network using any suitable communications protocol. Examples of the communications protocol include but may not be limited to Wi-Fi, Bluetooth RTM, radio frequency systems, infrared, LTE, GSM, GSM plus EDGE, CDMA, and quadband.

In an embodiment, the communications circuitry module 408 may be operative to create a communications network using any suitable communications protocol. For example, the communications circuitry module 408 may create a short-range communications network using a short-range communications protocol to connect to other devices. For example, the communications circuitry module 408 may be operative to create a local communications network using the Bluetooth, RTM protocol to couple the communication device 400 with a Bluetooth, RTM headset.

It may be noted that the computing device is shown to have only one communication operation; however, those skilled in the art would appreciate that the communication device 400 may include one more instances of the communications circuitry module 408 for simultaneously performing several communications operations using different communications networks. For example, the communication device 400 may include a first instance of the communications circuitry module 408 for communicating over a cellular network, and a second instance of the communications circuitry module 408 for communicating over Wi-Fi or using Bluetooth®.

In an embodiment, the same instance of the communications circuitry module 408 may be operative to provide for communications over several communications networks. In an embodiment, the communication device 400 may be coupled a host device for data transfers, synching the communications device, software or firmware updates, providing performance information to a remote source (e.g., providing riding characteristics to a remote server) or performing any other suitable operation that may require the communication device 400 to be coupled to a host device. Several computing devices may be coupled to a single host device using the host device as a server. Alternatively or additionally, the communication device 400 may be coupled to several host devices (e.g., for each of the plurality of the host devices to serve as a backup for data stored in the communication device 400).

While the disclosure has been presented with respect to certain specific embodiments, it will be appreciated that many modifications and changes may be made by those skilled in the art without departing from the spirit and scope of the disclosure. It is intended, therefore, by the appended claims to cover all such modifications and changes as fall within the true spirit and scope of the disclosure.

What is claimed is:

1. A method for monitoring intensity of pain experienced by one or more users, the method comprising:
assessing said intensity of pain experienced by each of said one or more users on one or more pain monitoring scales using one or more bio-markers, wherein said one or more users being pediatric patients;
fetching said one or more bio-markers associated with each of said one or more users by a plurality of bio-sensors;
determining a co-relation between said one or more bio-markers and said intensity of pain experienced by each of said one or more users; and
generating a pain profile for each of said one or more users, wherein said generated pain profile shows said intensity of pain experienced by each of said one or more users at various points in body aiding in treatment of each of said one or more users.

2. The method as recited in claim 1, wherein said one or more pain monitoring scales comprises at least one of a neonatal pain agitation and sedation scale (N-PASS), a pain assessment tool (PAT), a bernese pain scale for one or more neonates (BPSN), wong-baker scale, a face, legs, activity, crying and consolability (FLACC) scale, and an expert physician's quantified pain scale, wherein said one or more pain monitoring scales being configured to rank said intensity of pain experienced by at least one of one or more neonates, one or more infants and one or more toddlers.

3. The method as recited in claim 1, wherein said one or more pain monitoring scales comprises at least one of a visual analog scale (VAS), a verbal numerical rating scale (VNRS), a brief pain inventory (BPI), verbal descriptor scale (VDS), and the expert physician's quantified pain scale, or one or more pain scales based on subjective perception of pain experienced by each of said one or more users, wherein said one or more pain monitoring scales being configured to rank said intensity of pain experienced by pediatric population, said pediatric population comprises children being matured enough to interpret perception of the pain on each of the one or more pain monitoring scales.

4. The method as recited in claim 1, wherein said one or more bio-markers associated with each of said one or more users comprises at least one of heart rate (HR), heart rate variability (HRV), skin conductance, respiration information, blood pressure, photoplethysmography (PPG), oxygen saturation, electrocardiography (ECG), electroencephalography (EEG), muscle activity (EMG) and restlessness.

5. The method as recited in claim 1, wherein said plurality of bio-sensors comprises at least one of a finger based pulse oximeter, an accelerometer, a respiration monitor and a 1-lead disposable electrocardiography (ECG) patch and a multiple lead ECG sensing system.

6. The method as recited in claim 1, wherein said pain profile for each of said one or more users being generated using any combination of said plurality of bio-sensors.

7. The method as recited in claim 1, wherein said generated pain profile for each of said one or more users utilizes a pre-defined color coding based on said intensity and location of pain in the body of each of said one or more users, wherein a change in intensity of colors or quantification of pain being directly proportional to said intensity of pain experienced by said one or more users and assessed by one or more physicians.

8. The method as recited in claim 1, wherein said method distributes said one or more users based on phenotypical characteristics, genotypical characteristics and mental attributes.

9. The method as recited in claim 1, wherein said intensity of pain experienced by each of said one or more users being characterized by at least one of biological factors (gender and genetics), psychological factors (mood and attention), experimental factors, duration of measurement of said intensity of pain and location of each sensor of said plurality of bio-sensors on the body of each of said one or more users.

10. The method as recited in claim 1, further comprising tracking location of each of said one or more bio-sensors on the body of each of said one or more users for said monitoring of said intensity of pain experienced by each of said one or more users.

11. A system for monitoring intensity of pain experienced by one or more users, wherein said one or more users being pediatric patients, the system comprising:
a plurality of bio-sensors configured to fetch one or more bio-markers associated with each of said one or more users;
a pain monitoring application to monitor said intensity of pain experienced by said one or more users, wherein said pain monitoring application further comprises:
an input/output module configured to fetch said one or more bio-markers associated with each of said one or more users;
a display module configured to display said one or more bio-markers associated with said one or more users;
a diagnostic module configured to assess said one or more bio-markers of each of said one or more users to determine said intensity and location of pain of said one or more users;
a presentation module configured to generate a pain profile for each of said one or more users, wherein said generated pain profile shows said intensity of pain experienced by said one or more users at various points in body aiding in treatment of each of said one or more users; and
a database configured to store said fetched one or more bio-markers associated with each of said one or more users and said generated pain profile for each of said one or more users.

12. The system as recited in claim 11, wherein further comprising one or more pain monitoring scales being configured to rank said intensity of pain experienced by at least one of one or more neonates, one or more infants and one or more toddlers, wherein said one or more pain monitoring scales comprises at least one of a neonatal pain agitation and sedation scale (N-PASS), a pain assessment tool (PAT), a bernese pain scale for one or more neonates (BPSN), wong-baker scale, a face, legs, activity, crying, and consolability (FLACC) scale and an expert physician's quantified pain scale.

13. The system as recited in claim 12, wherein said one or more pain monitoring scales being configured to rank said intensity of pain experienced by pediatric population, said pediatric population comprises children being matured enough to interpret perception of the pain on each of the one or more pain monitoring scales, wherein said one or more pain monitoring scales comprises at least one of a visual analog scale (VAS), a verbal numerical rating scale (VNRS), a brief pain inventory (BPI), verbal descriptor scale (VDS), the expert physician's quantified pain scale or one or more pain scales based on subjective perception of pain experienced by each of said one or more users.

14. The system as recited in claim 11, wherein said diagnostic module is further configured to determine a co-relation between said one or more bio-markers associated with each of said one or more users and said intensity of pain experienced by each of said one or more users.

15. The system as recited in claim 11, wherein said diagnostic module is further configured to track location of each of said one or more bio-sensors on the body of each of said one or more users for said monitoring of said intensity of pain experienced by each of said one or more users.

16. The system as recited in claim 11, wherein said fetched one or more bio-markers associated with each of said one or more users comprises at least one of heart rate (HR), heart rate variability (HRV), skin conductance, respiration information, blood pressure, photoplethysmography(PPG), oxygen saturation, single or multiple lead electrocardiography (ECG), electroencephalography (EEG), muscle activity (EMG), pulse wave transit time, atrial kick, BCG (Balistocardiogram), EOG (Electrooculography), Dispersion based ECG, Impedence cardiography, GSR, VO₂ max, PaCO₂, facial features, stress, emotion detectors, cardiac output, oxygen saturation, blood glucose, blood gas, temperature, sweat, hydration, gaze, movements, and restlessness.

17. The system as recited in claim 11, wherein said generated pain profile for each of said one or more users utilizes a pre-defined color coding based on said intensity and location of pain in body of each of said one or more users, wherein a change in intensity of colors or quantification of pain being directly proportional to said intensity of pain experienced by said one or more users and assessed by one or more physicians.

18. The system as recited in claim 11, wherein said intensity of pain experienced by each of said one or more users being characterized by at least one of biological factors (gender and genetics), psychological factors (mood and attention), experimental factors, duration of measurement of said intensity of pain and location of each sensor of said plurality of bio-sensors on the body of each of said one or more users.

19. A computer system comprising:
one or more processors; and
a non-transitory memory containing instructions that, when executed by said one or more processors, causes said one or more processors to perform a set of steps, said set of steps comprising:
assessing said intensity of pain experienced by each of said one or more users on one or more pain monitoring scales by using one or more bio-markers;
fetching said one or more bio-markers associated with each of said one or more users by a plurality of bio-sensors; and
generating a pain profile of each of said one or more users, wherein said generated pain profile shows said intensity of pain experienced by each of said one or more users at various points in body aiding in treatment of each of said one or more users.

20. The computer system as recited in claim 19, wherein said non-transitory memory containing instructions that, when executed by said one or more processors, cause said one or more processors to perform a further step of determining a co-relation between said one or more bio-markers associated with each of said one or more users and said intensity of pain experienced by each of said one or more users.
